(19) United States
(12) Patent Application Publication
(10) Pub. No.: US 2005/0010262 A1
(43) Pub. Date: Jan. 13, 2005

(54) Modulation of the pain circuitry to affect chronic pain

(76) Inventors: Ali Rezai, Bratenhal, OH (US); Ashwini Sharan, Mt. Laurel, NJ (US)

Correspondence Address: KENYON & KENYON, 1500 K STREET, N.W., SUITE 700, WASHINGTON, DC, 20005, US

(21) Appl. No.: 10/502,349

(22) PCT Filed: Jan. 31, 2003
(86) PCT No.: PCT/US03/02846

Related U.S. Application Data
(60) Provisional application No. 60/353,697, filed on Feb. 1, 2002.

(51) Int. Cl. A61N 1/18

(52) U.S. Cl. 607/46

(57) ABSTRACT

The present invention relates to methods of affecting chronic pain by electrically and/or chemically stimulating target sites of the pain circuitry associated with chronic pain. Such target sites include cerebral target sites, including limbic structures, associated with the emotional and suffering components of chronic pain, as well as deep brain target sites associated with the affective and sensory components of chronic pain. Also provided is a method of affecting chronic pain by stimulating a target site of the pain circuitry associated with chronic pain to stimulate the synthesis or release of endogenous opioids.

### CROSS-REFERENCE TO RELATED APPLICATIONS

This application claims the benefit of Provisional U.S. Application No. 60/353,697, filed Feb. 1, 2002, which is incorporated by reference herein.

### BACKGROUND OF THE INVENTION

Chronic pain afflicts approximately 86 million Americans and it is estimated that United States business and industry loses about $90 billion dollars annually to sick time, reduced productivity, and direct medical and other benefit costs due to chronic pain among employees. Because of the staggering number of people affected by chronic pain, a number of therapies have been developed that attempt to alleviate the symptoms of this condition. Such therapies include narcotics, non-narcotics, analgesics, antidepressants, anticonvulsants, physical therapy, biofeedback, transcutaneous electrical nerve stimulation (TENS), as well as less conventional or alternative therapies. Other treatment options involve neuroaugmentive techniques such as spinal cord stimulation or intrathecal pumps. For a subset of patients, however, these therapies are inefficacious and more invasive procedures such as blocks, neurolysis and ablative procedures become the only options for treatment. In particular, ablative procedures, although infrequently utilized, are the primary alternative for patients unresponsive to other modes of treatment. Such procedures, however, have the fundamental limitation of being inherently irreversible and being essentially a “one-shot” procedure with little chance of alleviating or preventing potential side effects. In addition, there is a limited possibility to provide continuous benefits as the pathophysiology underlying the chronic pain progresses and the patient's symptoms evolve. Because of the inherent disadvantages of ablative procedures, electrical stimulation of the brain has become an attractive neurosurgical alternative to alleviate the symptoms of chronic pain.

Electrical stimulation of the brain for chronic pain has been used since the 1950s when temporary electrodes were implanted in the septal region for psychosurgery in patients with schizophrenia and metastatic carcinoma. In particular, electrodes were placed in the septum pellucidum in a region anterior and inferior to the foramen of Monro. In the 1960s, there were reports of stimulation of both the caudate nucleus and the septal region in six patients with intractable pain, but successful pain relief was obtained in only one patient. Despite these earlier reports of septal and caudate stimulation, current applications of electrical stimulation for pain involve thalamic, medial lemniscus, internal capsule stimulation, periventricular gray and pariaqueductal gray stimulation. For example, thalamic stimulation for pain relief was first reported for stimulation along the ventroposterolateral nucleus and ventralis posterior to relieve chronic intractable deafferentation pain and stimulation along the ventroposteromedial nucleus to relieve refractory facial pain. With respect to internal capsule stimulation, chronic stimulating electrodes have been implanted in the posterior limb of the internal capsule in a number of patients, including patients with lower-extremity pain and spasticity following spinal cord injury.

Although the above-mentioned target sites are all deep brain stimulation target sites, several studies have supported the role of motor cortex stimulation for pain control. For example, in the process of performing sensory cortex stimulation in an attempt to relieve thalamic pain, it was found that stimulation of the precentral gyrus/motor cortex was effective in relieving thalamic pain. Interestingly, stimulation of the sensory cortex exacerbated the pain in many patients.

Therefore, despite previous attempts to alleviate the symptoms of chronic pain by deep brain or cortical stimulation, there is still an unmet need for a method of treating chronic pain that is effective in a larger subset of the patient population.

### SUMMARY OF THE INVENTION

The present invention relates to a method of affecting chronic pain by electrically and/or chemically stimulating a target site of the pain circuitry involved in chronic pain to modulate the target site. In particular, one embodiment of the present invention provides a method of affecting chronic pain in a patient including implanting a stimulator in a target site of the brain and providing a stimulation signal to the stimulator to stimulate the target site to affect chronic pain. The target site is selected from the group consisting of the pre-frontal cortex, orbitofrontal cortex, anterior limb of the internal capsule, insular cortex, primary somatosensory cortex, secondary somatosensory cortex, cingulate cortex, anterior cingulate cortex, and posterior cingulate cortex, inferior frontal gyrus, middle frontal gyrus, superior frontal gyrus, medial frontal gyrus, parahippocampal gyrus, precuneus, amygdala, and hippocampus.

Another embodiment of the present invention provides a method of affecting chronic pain in a patient including implanting a stimulator in a target site of the brain and providing a stimulation signal to the stimulator to stimulate the target site. The target site is selected from the group consisting of the anterior nucleus of the thalamus, intralaminar thalamic nuclei, dorsomedial nucleus of the thalamus, mamillary body, lateral hypothalamus, locus coeruleus, dorsal raphe nucleus, substantia nigra pars compacta, substantia nigral pars reticulata, superior colliculus, tegmentum, ventral tegmentum, tectum, and medial thalamus, nucleus accumbens, ventral striatum, and ventral pallidum.

Another embodiment of the present invention provides a method of affecting chronic pain including implanting a stimulator in communication with a pain circuitry target site and providing a stimulation signal to the stimulator to stimulate the synthesis or release of an endogenous opioid to affect chronic pain.

### BRIEF DESCRIPTION OF THE FIGURES AND TABLES

FIG. 1 is a cross-sectional view of the brain showing placement of a stimulator to practice a method according to the present invention.

Table I provides cerebral target sites for affecting chronic pain and the corresponding stereotactic coordinates for these target sites.

Table II provides deep brain target sites for affecting chronic pain and the corresponding stereotactic coordinates for these target sites.

### DETAILED DESCRIPTION OF THE INVENTION

The present invention relates to methods of affecting chronic pain to regulate, prevent, treat, alleviate the symptoms of and/or reduce the effects of chronic pain. Although not wishing to be bound to any particular definition or characterization, chronic pain can generally be characterized as being nociceptive or non-nociceptive pain. Nociceptive pain, also referred to as somatic pain, involves direct activation of the nociceptors, such as mechanical, chemical, and thermal receptors, found in various tissues, such as bone, muscle, vessels, viscera, and cutaneous and connective tissue. The afferent somatosensory pathways are thought to be intact in nociceptive pain and examples of such pain include cancer pain from bone or tissue invasion, non-cancer pain secondary to degenerative bone and joint disease or osteoarthritis, and failed back surgery. The foregoing examples of nociceptive pain are in no way limiting and the methods of the present invention encompass methods of affecting all types of nociceptive pain.

Non-nociceptive pain, also referred to as neuropathic pain, or deafferentation pain, occurs in the absence of activation of peripheral nociceptors. Non-nociceptive pain often results from injury or dysfunction of the central or peripheral nervous system. Such damage may occur anywhere along the neuroaxis and includes thalamic injury or syndromes (also referred to as central pain, supraspinal central pain, or post-stroke pain); stroke; traumatic or iatrogenic trigeminal (trigeminal neuropathic) brain or spinal cord injuries; phantom limb or stump pain; postherpetic neuralgia; anesthesia dolorosa; brachial plexus avulsion; complex regional pain syndrome I and II; postcordotomy dysesthesia; and various peripheral neuropathies. The foregoing examples of non-nociceptive pain are in no way limiting and the methods of the present invention encompass methods of affecting all types of non-nociceptive pain.

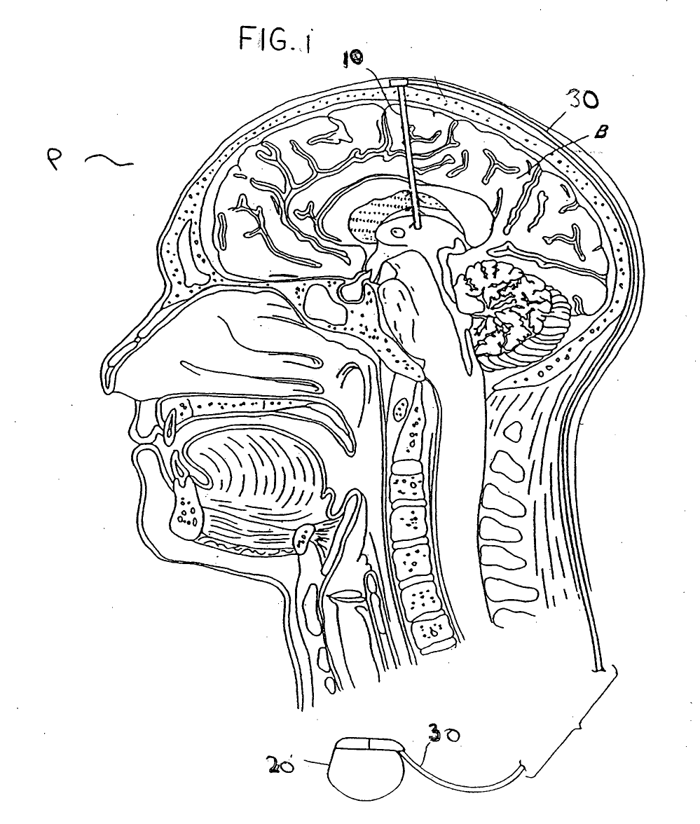

In general, the present invention provides for a method of affecting chronic pain by implanting a stimulator in a pain circuitry target site of the brain to modulate the target site such that chronic pain is affected. Referring to FIG. 1, in one example of a preferred mode of carrying out a method of the present invention, a stimulator 10, which can be either a catheter or electrode assembly, is implanted within a pain circuitry target site of brain B of a patient P. Stimulator 10 is, in turn, coupled to a stimulator controller 20, which is a pulse generator or drug pump, that generates electrical or chemical stimulation signals that are sent to stimulator 10 to electrically or chemically stimulate the pain circuitry target site. A connector 30, which is an insulated conductor in the case of electrical stimulation and an extension of a catheter in the case of chemical stimulation, couples stimulation controller 20 to stimulator 10. Stimulation controller 20 is, in turn, implanted in the chest, abdomen or any other part of a patient P's body and is preferably in patient P's control or is a radio frequency controlled device operated by an external transmitter. In the case of a chemical delivery system where stimulator 10 is a catheter, stimulation controller 20 is preferably accessed subcutaneously such that a hypodermic needle can be inserted through the skin to inject a quantity of a chemical agent, such as a neuromodulation agent. The chemical agent is delivered from the stimulation controller 20 through a catheter port into the stimulator 10. Stimulation controller 20 may be a permanently implanted in patient P or only temporarily implanted such as the temporary neurostimulator described in co-pending U.S. Provisional No. 60/358,176.

The methods of the present invention generally include implanting a stimulator in a pain circuitry target site and providing a stimulation signal to the stimulator to stimulate the pain circuitry target site. By “pain circuitry target site” is meant either a cerebral target site or a deep brain target site, as described by the present invention. Referring to Table I, cerebral target sites according to the present invention are the pre-frontal cortex, orbitofrontal cortex, anterior cingulate cortex, posterior cingulate cortex, insular cortex, primary somatosensory cortex, secondary somatosensory cortex, inferior frontal gyrus, middle frontal gyrus, superior frontal gyrus, medial frontal gyrus, parahippocampal gyrus, precuneus, amygdala, and hippocampus. Table I also provides the x, y, and z, coordinates of these cerebral target sites, relative to the anterior commissure-posterior commissure line, unless otherwise indicated. As will be readily appreciated by one of skill in the art, targeting of the insular, primary and secondary somatosensory cortex can be achieved by standard neuronavigational techniques which identify standard surface landmarks on the brain.

TABLE I

| | Lateral | AP | Sagittal |
|---|---|---|---|
| Cerebral Target Site | (X) | (Y) | (Z) |
| Pre-frontal Cortex | Falx to | 20 mm anterior | Superior, |
| | Sphenoid | to coronal | middle, and |
| | Ridge | suture and | inferior |
| | | anteriorly | frontal gyrus |
| Orbitofrontal cortex | Medial to inferior | Anterior | Frontal fossa |
| | frontal gyrus and | commissure | base to |
| | lateral to gyrus | and anteriorly | cingulate |
| | rectus | | sulcus |
| Anterior Limb of the | 10 to 20 | AC: 3 to 10 | 10 to 0 |
| Internal Capsule | | | |
| Cingulate Cortex | 5 to 9 | 15 to 25 | 1 to 5 above |
| | | posterior to | ventricular |
| | | frontal hip tip | roof |
| Anterior Cingulate | 0 to 13 | 0 to 55 | 26 to 38 |
| Cortex | | | |
| Posterior Cingulate | 0 to 14 | 0 to −37 | 22 to 36 |
| Cortex | | | |
| Inferior Frontal Gyrus | 33 to 59 | 7.7 to 55 | 5 to 31 |
| Middle Frontal Gyrus | 25 to 49 | −17 to 72 | 31 to 67 |
| Superior Frontal Gyrus | 0 to 26 | −19 to 75 | 36 to 67 |
| Medial Frontal Gyrus | 0 to 19.5 | 22 to 78 | −10 to 0 |
| Parahippocampal | 16 to 29 | 7.67 to −29 | −27 to −6 |
| Gyrus | | | |
| Precuneus | 0 to 18 | −37 to −69 | 7.6 to 63 |
| Amygdala | 12 to 22 | MCP: 3 to 15 | −15 to −25 |
| Hippocampus | Medial to | Amygdala to | −10 to −25 |
| | temporal horn | 30 posterior | |
| Nucleus Accumbens | 5 to 13 | AC: 0 to 5 | 3 to −5 |
| Ventral Striatum | 15 to 30 | MCP: 0 to 10 | 3 to 10 |
| Ventral Pallidum | 15 to 30 | MCP: 0 to 6 | 3 to 10 |

All measurements in millimeters
MCP: Relative to midcommisural point (anterior is positive)
AC: Relative to anterior commisure (anterior is positive)
PC: Relative to the posterior commisure (anterior is positive)
Sagittal: Superior is positive, inferior is negative

Therefore, in one embodiment, the present invention provides a method of affecting chronic pain by implanting a stimulator in a cerebral target site and providing a stimulation signal to the stimulator to stimulate the cerebral target site to affect chronic pain. Although the present invention contemplates the stimulation of any one or any combination of cerebral target sites, the particular cerebral target sites can be chosen as a function of the particular effect desired to be achieved. For example, without wishing to be bound by theory, if the emotional, suffering, and motivational components of a patient's chronic pain are desired to be alleviated, then the limbic structures including the hippocampus, parahippocampal gyrus, cingulate cortex, and/or the amygdala may be stimulated. If the sensory or discriminatory aspects of pain relay are desired to be alleviated then the primary somatosensory cortex, secondory somatosensory cortex, and/or the insular cortex may be stimulated.

Referring to Table II, deep brain targets according to the present invention are the anterior nucleus of the thalamus, intralaminar thalamic nuclei, dorsomedial nucleus of the thalamus, locus coeruleus, mammillary bodies, lateral hypothalamus, substantia nigra pars compacta, substantia nigra pars reticulata, superior colliculus, tegmentum, ventral tegmentum, tectum, medial thalamus, nucleus accumbens, ventral striatum, and ventral pallidum. Preferred intralaminar thalamic nuclei according to the present invention include the anterior, posterior, and midline intralaminar nuclei. Preferred anterior intrathalamic nuclei include the central lateral, paracentralis, and paralamellar nuclei. Preferred posterior intralaminar nuclei include the centromedian and parafasicularis nuclei. Preferred midline intralaminar nuclei include the paraventricularis and central medial nuclei. Table II also provides x, y, and z coordinates of these deep brain target sites, relative to the anterior commissure-posterior commissure line, unless otherwise indicated.

TABLE II

| | Lateral | AP | Sagittal |
|---|---|---|---|
| Deep Brain Target Site | (X) | (Y) | (Z) |
| Anterior Nucleus of | 0 to 10 | AC: 0 to 10 | 10 to −10 |
| Thalamus | | | |
| Anterior Intralaminar | 7 to 13 | MCP to 10 anterior | 0 to 13 |
| Nuclei | | | |
| Posterior Intralaminar | 5 to 10 | MCP: −5 to PC: −7 | 0 to 13 |
| Nuclei | | | |
| Midline Intralaminar | 2 to 8 | MCP to 10 anterior | 0 to 13 |
| Nuclei | | | |
| Dorsomedial Nucleus of | 0 to 10 | AC: 0 to −5 | 0 to 13 |
| Thalamus | | | |
| Mammillary Body | 0 to 5 | MCP: 3 to 15 | −15 to −25 |
| Lateral Hypothalamus | 5 to 15 | AC 5 to −5 | 0 to −10 |
| Locus Coeruleus | 0 to 7 | MCP: −10 to −20 | −5 to −20 |
| Dorsal Raphe Nucleus | 0 to 7 | MCP: −10 to −20 | −3 to −15 |
| Substantia Nigra Pars | 5 to 12 | MCP: 5 to −12 | −5 to −20 |
| Compacta | | | |
| Substantia Nigra Pars | 6 to 15 | MCP: 5 to −12 | −5 to −20 |
| Reticulata | | | |
| Superior Colliculus | 0 to 12 | PC: −5 to −15 | 0 to −7 |
| Tectum | 0 to 12 | −5 to −15 | 0 to −7 |
| Tegmentum | 0 to 12 | −5 to −10 | 0 to −7 |
| Ventral Tegmentum | 0 to 15 | MCP: 3 to −10 | −5 to −20 |
| Medial Thalamus | 0 to 26 | −19 to 75 | 36 to 67 |

All measurements in millimeters
MCP: Relative to midcommisural point (anterior is positive)
AC: Relative to anterior commisure (anterior is positive)
PC: Relative to the posterior commisure (anterior is positive)
Sagittal: Superior is positive, inferior is negative

Therefore, in another embodiment, the present invention provides a method of affecting chronic pain by implanting a stimulator in a deep brain target site and providing a stimulation signal to the stimulator to stimulate the deep brain target site to affect chronic pain. Similar to the method of the present invention directed to stimulating cerebral target sites, the present invention contemplates the stimulation of any one or any combination of deep brain target sites. However, particular deep brain target sites can be chosen as a function of the particular effect desired to be achieved. For example, without wishing to be bound by theory, if the emotional, suffering, and motivational components of a patient's chronic pain are desired to be alleviated, then the limbic structures, for example, such as the locus coeruleus, lateral hypothalamus, mammillary bodies, and/or anterior thalamic nuclei may be stimulated. If the affective aspects of pain relay are desired to be alleviated then the intralaminar thalamic nuclei may be stimulated.

Although the stereotactic coordinates for the aforementioned pain circuitry target sites have been provided, the exact location of the target site may vary from patient to patient. Accordingly, standard neurological procedures can be used to localize the x, y, and z coordinates of the target site in a specific patient. For example, a CT scan, an MRI scan, and computerized standard brain atlas can be used to create a 3-dimensional image of a patient's brain and within that image the x, y, and z, coordinates can be identified.

In another embodiment of the present invention, a method of affecting chronic pain includes implanting a stimulator in communication with a pain circuitry target site and providing a stimulation signal to the stimulator to stimulate the synthesis or release of an endogenous opioid to affect chronic pain. Non-limiting examples of endogenous opioids include beta endorphin and metenkephalin. In a preferred embodiment, the pain circuitry target site is the locus coeruleus or the intralaminar thalamic nuclei, including the centromedian, parafasicularis, and the central lateral nuclei. Although not wishing to be bound by theory, by implanting a stimulator in communication with a pain circuitry target site to stimulate the synthesis or release of an endogenous opioid, it is intended to modulate the endogenous analgesia pathway, which is thought to include the periaqueductal gray, the nucleus raphe magnus, the locus coeruleus, and the magnocellular part of the nucleus reticularis gigantocellularis. These pathways are also thought to involve descending projections from the midbrain to the dorsal horn as well as various intralaminar nuclei and medial nuclei.

Although this embodiment of the present invention contemplates electrical and/or chemical stimulation to stimulate the synthesis or release of an endogenous opioid to affect chronic pain, this embodiment is particularly useful for chemical stimulation as chemical agents can be delivered directly to the pain circuitry target site. Such chemical agents include antagonists, agonists, other therapeutic neuromodulation agents and any combinations thereof that bind to the receptors of endogenous opioids to regulate the actions of the receptors. Although such chemical agents are generally administered orally in traditional pharmacotherapies, by directly stimulating the target sites in the brain that are modulated by such opioids, low and precise doses of the chemical agents can be administered so as to minimize or avoid the side effects and delayed onset of relief common to traditional pharmacotherapy.

With respect to particular details of chemical stimulation according to the present invention, whether employed alone or in combination with electrical stimulation, once the stimulator (i.e. a catheter) is secured in place in the pain circuitry target site, the stimulation controller (i.e. drug pump) is activated thereby delivering a chemical agent to the target site. The chemical agent may be a neurotransmitter mimick; neuropeptide; hormone; pro-hormone; antagonist, agonist, reuptake inhibitor, or degrading enzyme thereof; peptide; protein; therapeutic agent; amino acid; nucleic acid; stem cell or any combination thereof and may be delivered by a slow release matrix or drug pump. In a preferred embodiments, the chemical agent is an antagonist/agonist of an inhibitory neurotransmitter, such as GABA; an excitatory amino acid, such as adenosine; an excitatory neurotransmitter, such as dopamine or glutamate; and/or a neuropeptide, such as substance P. Examples of therapeutic agents include lidocaine, morphine, gabapentin, clonidine, muscimol, or any agents within similar families thereof and any combination of these therapeutic agents. The chemical agents may also be delivered continuously or intermittently.

With respect to particular details of electrical stimulation according to the present invention, once the stimulator (i.e. electrode) is secured in place in the pain circuitry target site, the stimulation controller (i.e. pulse generator) is activated thereby applying to the target site an oscillating electrical signal having specified pulsing parameters. The oscillating electrical signal may be applied continuously or intermittently and the pulsing parameters, such as the pulse width, amplitude, frequency, voltage, current, intensity, and/or waveform may be adjusted to achieve affect a desired result. Preferably, the oscillating electrical signal is operated at a voltage between about 0.1 μV to about 20 V. More preferably, the oscillating electrical signal is operated at a voltage between about 1 V to about 15 V. Preferably, the electric signal is operated at a frequency range between about 2 Hz to about 2500 Hz. More preferably, the electric signal is operated at a frequency range between about 2 Hz to about 200 Hz. Preferably, the pulse width of the oscillating electrical signal is between about 10 microseconds to about 1,000 microseconds. More preferably, the pulse width of the oscillating electrical signal is between about 50 microseconds to about 500 microseconds. The waveform may be, for example, biphasic square wave, sine wave, or other electrically safe and feasible combination. Preferably, the application of the oscillating electrical signal is: monopolar when the electrode is monopolar, bipolar when the electrode is bipolar, and multipolar when the electrode is multipolar.

The present invention contemplates either chemical or electrical stimulation and both electrical and chemical stimulation of a pain circuitry target site to affect chronic pain. One non-limiting example of the use of chemical and electrical stimulation to affect chronic pain, particularly when such chronic pain is characterized by cellular damage at the pain circuitry target site, involves repopulating the target site with undifferentiated cells or nucleic acids and stimulating the growth of such cells or replication of such nucleic acids by electrical stimulation. Such repopulation of cells can be carried out using a cellular or molecular approach. Cellular approaches involve injecting or infusing undifferentiated cells, which are preferably cultured autologous cells, into the target site. Molecular approaches involve injecting or infusing nucleic acids, whether in the form of naked or plasmid DNA, into the target site. Methods of delivering nucleic acids to a cellular target site are well known in the art and generally involve the use of delivery vehicles such as expression vector or liposomes. Non-limiting examples of expression vectors for use in this embodiment of the present invention include bacterial expression vectors and viral expression vectors such as retroviruses, adenoviruses, or adeno-associated viral vectors.

In the case of repopulating the target site with nucleic acid molecules, such molecules are preferably recombinant nucleic acid molecules and can be prepared synthetically or, preferably, from isolated nucleic acid molecules, as is known in the art. A nucleic acid is “isolated” when it is purified away from other cellular constituents, such as, for example, other cellular nucleic acids or proteins by standard techniques known to those of skill in the art. The coding region of the nucleic acid molecule can encode a full length gene product or a fragment thereof or a novel mutated or fusion sequence. The coding sequence can be a sequence endogenous to the target cell, or exogenous to the target cell. The promoter, with which the coding sequence is operably associated, may or may not be one that normally is associated with the coding sequence.

The cellular or genetic material can be delivered simultaneously with the electrical stimulation, or the cellular or genetic material can be delivered separately. One particularly advantageous feature of this embodiment of combined chemical and electrical stimulation is that the expression of the nucleic acid molecules may be regulated by electrical stimulation. Namely, the amplitude, intensity, frequency, duration and other pulsing parameters may be used to selectively control expression of nucleic acid molecules delivered to the target site. Further details of the use of electrical stimulation and nucleic acid delivery to repopulate a target site are described in U.S. Pat. No. 6,151,525, which describes the use of electrical current to modify contractile cells to form new contractile tissue and which is incorporated by reference herein.

Another example of electrical and chemical stimulation being used together, is the use of electrical stimulation to modulate the expression of cellular receptors at the target site.

Notwithstanding whether chemical and/or electrical stimulation is employed in the methods of the present invention, the present invention also contemplates the use of a closed-loop feedback mechanism in conjunction with chemical or electrical stimulation. In such an embodiment, a pain circuitry target site is stimulated in response to a detected bodily activity associated with chronic pain. In particular, this embodiment includes implanting a stimulator in communication with a pain circuitry target site, detecting a bodily activity of the body associated with the pain circuitry target site, and providing a stimulation signal to a stimulator in response to the detected bodily activity to stimulate the target site to affect chronic pain. Such bodily activity to be detected is any characteristic or function of the body, and includes, for example, respiratory function, body temperature regulation, blood pressure, metabolic activity, cerebral blood flow, pH levels, vital signs, galvanic skin responses, perspiration, electrocardiogram, electroencephalogram, action potential conduction, chemical production, body movement, and response to external stimulation. For example, in a preferred embodiment, a patient's threshold to pain could be measured prior to stimulation of the pain circuitry target site and then the patient's threshold to pain could be measured during stimulation of the pain circuitry target site through the use of tactile stimulation or exposure to noxious stimuli to determine the stimulation signal. In addition or alternatively, the patient's threshold to increases or decreases in temperature could be measured during stimulation of the pain circuitry target site to determine the stimulation signal.

In another embodiment of the present invention, the bodily activity of the body includes an electrical or chemical activity of the body and may be detected by sensors located on or within the body. For example, such activity may be detected by sensors located within or proximal to the target site, distal to the target site but within the nervous system, or by sensors located distal to the target site outside the nervous system. Examples of electrical activity detected by sensors located within or proximal to the target site include sensors that measure neuronal electrical activity, such as the electrical activity characteristic of the signaling stages of neurons (i.e. synaptic potentials, trigger actions, action potentials, and neurotransmitter release) at the target site and by afferent and efferent pathways and sources that project to and from or communicate with the target site. For example, if the target site is an intralaminar thalamic nuclei, then sensors can measure, at any signaling stage, neuronal activity of the intralaminar thalamic nuclei and the medial part of the spinothalamic tract, the spinoreticular formation, and the spinomesencephalic tract. In particular, the sensors may detect the rate and pattern of the neuronal electrical activity to determine the stimulation signal to be provided to the stimulator.

Examples of chemical activity detected by sensors located within or proximal to the target site include sensors that measure neuronal activity, such as the modulation of neurotransmitters, hormones, pro-hormones, neuropeptides, peptides, proteins, electrolytes, endogenous opioids, or small molecules by the target site and modulation of these substances by afferent and efferent pathways and sources that project to and from the target site or communicate with the target site.

With respect to detecting electrical or chemical activity of the body by sensors located distal to the target site but still within the nervous system, such sensors could be placed in the brain, the spinal cord, cranial nerves, and/or spinal nerves. Sensors placed in the brain are preferably placed in a layer-wise manner in the direction of increasing proximity to the target site. For example, a sensor could be placed on the scalp (i.e. electroencephalogram), in the subgaleal layer, on the skull, in the dura mater, in the sub dural layer and in the parenchyma (i.e. in the frontal lobe, occipital lobe, parietal lobe, temporal lobe) to achieve increasing specificity of electrical and chemical activity detection. The sensors could measure the same types of chemical and electrical activity as the sensors placed within or proximal to the target site as described above.

With respect to detecting electrical or chemical activity by sensors located distal to the target site outside the nervous system, such sensors may be placed in venous structures and various organs or tissues of other body systems, such as the endocrine system, muscular system, respiratory system, circulatory system, urinary system, integumentary system, and digestive system or such sensors may detect signals from these various body systems. For example, with respect to the respiratory system, sensors could detect lung function such as signs of hyperventilation as a measurement of chronic pain; with respect to the circulatory system, sensors could detect leg discoloration, as a measurement of chronic pain; with respect to the integumentary system, sensors could detect perspiration or response to tactile stimulation as a measurement of chronic pain; with respect to the muscular system, sensors, such as accelorometers, could detect physical activity of the body such as head movements. All the above-mentioned sensing systems may be employed together or any combination of less than all sensors may be employed together.

After the sensor(s) detect the relevant bodily activity associated with the pain circuitry target site, the sensors generate a sensor signal. The sensor signal is processed by a sensor signal processor and provides a control signal to the stimulation controller, which is a signal generator or drug pump depending on whether electrical or chemical stimulation is desired. The stimulation controller, in turn, generates a response to the control signal by providing a stimulation signal to the stimulator. The stimulator then stimulates the target site to affect chronic pain. In the case of electrical stimulation, the control signal may be an indication to initiate, terminate, increase, decrease or change the rate or pattern of a pulsing parameter of the electrical stimulation and the stimulation signal can be the respective initiation, termination, increase, decrease or change in rate or pattern of the respective pulsing parameter. In the case of chemical stimulation, the control signal can be an indication to initiate, terminate, increase, decrease or change the rate or pattern of the amount or type of chemical agent administered, and the stimulation signal can be the respective initiation, termination, increase, decrease or change in the rate or pattern of the amount or type of chemical agent administered. The processing of closed-loop feedback systems for electrical and chemical stimulation are described in more detail in respective U.S. Pat. Nos. 6,058,331 and 5,711,316, both of which are incorporated by reference herein.

Although the application of sensors to detect bodily activity are within the scope and spirit of the present invention, the present invention also contemplates the relevant bodily activity to be detected without sensors. For example, signs of hyperventilation and leg discoloration, as well as visual analogs and pain scores can be made or analyzed by visual observation without the assistance of sensors. In such case the stimulation signal could still be an initiation, termination, increase, decrease, or change in the rate or pattern of electrical and/or chemical stimulation in response to the visual observation.

Although not wishing to be bound by the description of a particular procedure, one exemplary procedure effectuating the methods of the present invention shall now be described with respect to electrical stimulation of a pain circuitry target site. Generally, the procedure begins with the patient having a stereotactic head frame mounted to the patient's skull, although frameless techniques may also be used. The patient then typically undergoes a series of MRI and/or CT sessions, during which a series of two dimensional slice images of the patient's brain are built up into a quasi-three dimensional map in virtual space. This map is then correlated to the three dimensional stereotactic frame of reference in the actual surgical field. In order to align these two coordinate frames, both the instruments and the patient should be situated in correspondence to the virtual map. A current method of achieving this alignment is to rigidly mount to the head frame to the surgical table. Subsequently, a series of reference points are established relative to aspects of the frame and patient's skull, so that a computer can adjust and calculate the correlation between the actual surgical field of the patient's head and the virtual space model of the patient's brain MRI scans. Initial anatomical localization of the pain circuitry target site is achieved either directly using the MRI images, or indirectly using interactive anatomical atlas programs that map the atlas image onto the stereotactic image of the brain. This indirect targeting approach involves entering the stereotactic anterior commissure (AC) and posterior commissure (PC) coordinates into a computer with a commercially available program containing digitized diagrams of sagittal brain sections from a standardized brain atlas. The program transcribes the patient's calculated AC-PC intercommissural line onto the digitized map at the sagittal laterality of interest. On these maps, the pain circuitry targets sites can be localized. The subsequently generated map is overlaid onto a millimeter grid ruled in stereotactic coordinates in the anteroposterior and dorsoventral scales with a corresponding diagram of the brain nuclei and tracts depicted in the chosen laterality. The laterality of the maps is chosen according to the location of the pain. Typical laterality is 12 to 14 millimeters from the midline for facial pain, 14 to 15 mm for upper extremity pain, and 15 to 17 millimeters for lower-extremity pain.

Another method of localizing the pain circuitry target site involves the fusion of functional and structural medical imaging. Such methods for localizing targets in the body and guiding diagnostic or therapeutic instruments toward a target region in the body have been described in U.S. Pat. No. 6,368,331, issued on Apr. 9, 2002 to Front et al., U.S. patent application Publication Ser. No. 2002/0032375, published Mar. 14, 2002 by Bauch et al., and U.S. patent application Publication Ser. No. 2002/0183607, published Dec. 5, 2002 by Bauch et al., all of which are hereby incorporated by reference in their entireties. Methods for target localization specifically within the nervous system, including the brain, have been described in U.S. Provisional Application No. 60/353,695, filed Feb. 1, 2002, by Rezai et al. which is hereby incorporated by reference in its entirety. Specifically, provided in U.S. Provisional Application No. 60/353,695 is a method of medical imaging, comprising: placing a fiducial marker proximate to an area of a body to be imaged; obtaining a first image of the area of the body using a first medical imaging technique, the first image including a first image of the fiducial marker; obtaining a second image of the area of the body using a second medical imaging technique, the second image including a second image of the fiducial marker, the second medical imaging technique being different than the first medical imaging technique; superimposing the first image of the area of the body and the second image of the area of the body; and aligning the first image of the first fiducial marker with the second image of the fiducial marker. Useful medical imaging techniques to obtain functional images include but are not limited to functional MRI, PET or MEG. Useful medical imaging techniques to obtain structural images include but are not limited to volumetric MRI and CT.

Subsequent to the stereotactic imaging (or finctional and structural imaging), acquisition of the images, and anatomical localization, the patient is taken to the operating room. The surgery can be performed under either local or general anesthetic, but preferably under local anesthesia in order to allow communication with the patient. An initial incision is made in the scalp, preferably 2.5 centimeters lateral to the midline of the skull, anterior to the coronal suture. A burr hole is then drilled in the skull itself; the size of the hole being suitable to permit surgical manipulation and implantation of an electrode. This size of the hole is generally about 14 millimeters. The dura is then opened, and fibrin glue is applied to minimize cerebral spinal fluid leaks and the entry of air into the cranial cavity. A guide tube cannula with a blunt tip is then inserted into the brain parenchyma to a point approximately one centimeter from the target tissue. At this time physiological localization starts with the ultimate aim of correlating the anatomical and physiological findings to establish the final stereotactic target structure.

Physiological localization using single-cell microelectrode recording is preferably performed for definitively identifying the pain circuitry target site by neuronal firing patterns of individual neurons. Single-cell microelectrode recordings obtained from intralaminar thalamic cells typically have a characteristic bursting activity. In addition to microelectrode recording, microstimulation and or macrostimulation may be performed to provide further physiological localization.

Once the final pain circuitry target site has been identified in the actual spatial frame of reference, the electrode is inserted into the target site and a hand-held pulse generator (Screener) is used for intraoperative test stimulation. Various pole combinations and stimulation frequency, pulse width, and intensity are used to determine the thresholds for therapeutic and adverse effects. Thereafter the electrode is locked into the burr hold ring to prevent lead migration. The proximal portion of the electrode is then attached to a transcutaneous pacing wire for a test trial period. After the test period, the patient undergoes implantation of a pulse generator or radio-frequency-coupled receiver.

Implanting the pulse generator is generally carried out with the patient under general anesthesia. The pulse generator is implanted in the infraclavicular space by tunneling from the frontal inicision to the infraclavicular space. The pulse generator can be powered by a battery and can be activated externally by an external transmitter.

Although the invention has been described with reference to the preferred embodiments, it will be apparent to one skilled in the art that variations and modifications are contemplated within the spirit and scope of the invention. The figures, tables, and description of the preferred embodiments are made by way of example rather than to limit the scope of the invention, and it is intended to cover within the spirit and scope of the invention all such changes and modifications.

1. A method of affecting chronic pain in a patient comprising:
a) implanting a stimulator in a target site of the brain; and
b) providing a stimulation signal to the stimulator to stimulate the target site to affect chronic pain, the target site selected from the group consisting of the pre-frontal cortex, orbitofrontal cortex, anterior limb of the internal capsule, insular cortex, primary somatosensory cortex, secondary somatosensory cortex, cingulate cortex, anterior cingulate cortex, and posterior cingulate cortex, inferior frontal gyrus, middle frontal gyrus, superior frontal gyrus, medial frontal gyrus,parahippocampal gyrus, precuneus, amygdala, and hippocampus.

2. The method of claim 1, wherein the target site is the pre-frontal cortex.

3. The method of claim 1, wherein the target site is the orbitofrontal cortex.

4. The method of claim 1, wherein the target site is the anterior limb of the internal capsule.

5. The method of claim 1, wherein the target site is the insular cortex.

6. The method of claim 1, wherein the target site is the primary somatosensory cortex.

7. The method of claim 1, wherein the target site is the seconary somatosensory cortex.

8. The method of claim 1, wherein the target site is the cingulate cortex.

9. The method of claim 1, wherein the target site is the anterior cingulate cortex.

10. The method of claim 1, wherein the target site is the posterior cingulate cortex.

11. The method of claim 1, wherein the target site is the inferior frontal gyrus.

12. The method of claim 1, wherein the target site is the middle frontal gyrus.

13. The method of claim 1, wherein the target site is the superior frontal gyrus.

14. The method of claim 1, wherein the target site is the medial frontal gyrus.

15. The method of claim 1, wherein the target site is the parahippocampal gyrus.

16. The method of claim 1, wherein the target site is the precuneus.

17. The method of claim 1, wherein the target site is the amygdala.

18. The method of claim 1, wherein the target site is the hippocampus.

19. A method of affecting chronic pain in a patient comprising:
a) implanting a stimulator in a target site of the brain; and
b) providing a stimulation signal to the stimulator to stimulate the target site, the target site selected from the group consisting of the anterior nucleus of the thalamus, intralaminar thalamic nuclei, dorsomedial nucleus of the thalamus, mammillary body, lateral hypothalamus, locus coeruleus, dorsal raphe nucleus, substantia nigra pars compacta, substantia nigral pars reticulata, superior colliculus, tegmentum, ventral tegmentum, tectum, medial thalamus, nucleus accumbens, ventral striatum, and ventral pallidum.

20. The method of claim 19, wherein the target site is the anterior nucleus of the thalamus.

21. The method of claim 19, wherein the target site is the intralaminar thalamic nuclei.

22. The method of claim 19, wherein the target site is the dorsomedial nucleus of the thalamus.

23. The method of claim 19, wherein the target site is the mammillary body.

24. The method of claim 19, wherein the target site is the lateral hypothalamus.

25. The method of claim 19, wherein the target site is the locus coeruleus.

26. The method of claim 19, wherein the target site is the dorsal raphe nucleus.

27. The method of claim 19, wherein the target site is the substantia nigra pars compacta.

28. The method of claim 19, wherein the target site is the substantia nigra pars reticulata

29. The method of claim 19, wherein the target site is the superior colliculus.

30. The method of claim 19, wherein the target site is the tegmentum.

31. The method of claim 19, wherein the target site is the ventral tegmentum.

32. The method of claim 19, wherein the target site is the tectum.

33. The method of claim 19, wherein the target site is the ventral thalamus.

34. The method of claim 19, wherein the target site is the nucleus accumbens.

35. The method of claim 19, wherein the target site is the ventral striatum.

36. The method of claim 19, wherein the target site is the ventral pallidum

37. A method of affecting chronic pain comprising:
a) implanting a stimulator in communication with a pain circuitry target site; and
b) providing a stimulation signal to the stimulator to stimulate the synthesis or release of an endogenous opioid to affect chronic pain.

38. A method of affecting chronic pain comprising:
a) implanting a stimulator in communication with a pain circuitry target site;
b) detecting a bodily activity of the body associated with the chronic pain;
c) providing a stimulation signal to the stimulator in response to the detected bodily activity; and
d) stimulating the target site to affect the hypothalamic-related condition

39. Use of a stimulator adapted to be implanted in a target site and provided with a stimulation signal to stimulate the target site to affect chronic pain, wherein the target site is selected from the group consisting of pre-frontal cortex, orbitofrontal cortex, anterior limb of the internal capsule, insular cortex, primary somatosensory cortex, secondary somatosensory cortex, cingulate cortex, anterior cingulate cortex, and posterior cingulate cortex, inferior frontal gyrus, middle frontal gyrus, superior frontal gyrus, medial frontal gyrus, parahippocampal gyrus, precuneus, amygdala, hippocampus, nucleus accumbens, ventral striatum, ventral pallidum, anterior nucleus of the thalamus, intralaminar thalamic nuclei, dorsomedial nucleus of the thalamus, mammillary body, lateral hypothalamus, locus coeruleus, dorsal raphe nucleus, substantia nigra pars compacta, substantia nigral pars reticulata, superior colliculus, tegmentum, ventral tegmentum, tectum, and medial thalamus.
